(19) United States
(12) Patent Application Publication
(10) Pub. No.: US 2007/0082905 A1
(43) Pub. Date: Apr. 12, 2007

(54) PHARMACEUTICAL COMPOSITIONS COMPRISING N-TRIAZOLYMETHYL-PIPERAZINE COMPOUNDS AND METHODS OF USING SAME

(76) Inventors: Michiel Henricus De Vries, Weesp (NL); Marianne de Bruijn, Weesp (NL)

Correspondence Address: MAYER, BROWN, ROWE & MAW LLP, P.O. BOX 2828, CHICAGO, IL, 60690-2828, US

(21) Appl. No.: 11/419,706

(22) Filed: May 22, 2006

Related U.S. Application Data
(60) Provisional application No. 60/685,458, filed on May 27, 2005.

(51) Int. Cl. A61K 31/5377; A61K 31/496

(52) U.S. Cl. 514/235.5; 514/254.05

(57) ABSTRACT

The present invention relates to N-triazolylmethyl-piperazine compounds, to pharmaceutical compositions comprising such compounds, and to the use of such compositions in treating and preventing diseases and disorders.

This application claims priority to U.S. Provisional Patent Application Ser. No. 60/685,458 filed on May 27, 2005, the entirety of which is incorporated by reference herein.

### FIELD OF THE INVENTION

In various embodiments, the present invention relates to 2-indolylmethyl-piperazine derivative compounds, to pharmaceutical compositions comprising such compounds, and to the use of such compositions in treating and preventing diseases and disorders.

### BACKGROUND OF THE INVENTION

Neurokinin-1 (NK-1) receptors have been identified in the central nervous system as well as in peripheral organs including the gastrointestinal and respiratory system, the genitourinary tract, and the vascular endothelium. The undecapeptide, Substance P, a member of the tachykinin family, is the natural agonist with the highest affinity to the NK-1 receptor, and is a mediator of emesis, pain transmission, neurogenic inflammation, and endothelium-dependent vasodilation. Furthermore, Substance P mediates the transmission of afferent perceptional signals from the gastrointestinal tract and mediates neuromuscular transmission in the enteric nervous system, resulting in the activation of gastrointestinal motility.

U.S. Pat. No. 6,407,106 discloses compounds with neurokinin receptor antagonistic properties, which are suitable for treating peripheral disturbances such as functional and inflammatory disturbances of the gastrointestinal tract. Furthermore, compounds that are structurally similar are provided in WO 98/57954, which compounds are ascribed general properties of antagonizing tachykinin, neurokinin A or alternatively neurokinin B and which are capable of influencing the central nervous system (CNS).

2-Indolylmethyl-piperazine derivatives having a different substitution pattern from the compounds of the present invention are described in EP 0 899 270 A1.

Additional 2-indolylmethyl-piperazine derivatives having a different substitution pattern from the compounds of the present invention are described in EP 0 655 442 A1.

### SUMMARY OF THE INVENTION

In various embodiments, the present invention provides 2-indolylmethyl-piperazine derivatives that are antagonistic to neurokinin receptors and that are substituted at a nitrogen of the piperazine parent structure by a triazolylmethyl radical. In other embodiments, the invention provides pharmaceutical compositions comprising such compounds. In still other embodiments, the invention provides methods for using compounds and compositions of the invention in the treatment and prevention of diseases and disorders.

### BRIEF DESCRIPTION OF THE DRAWINGS

FIG. 1 shows a semilogarithmic plot of plasma concentrations of the NK-1 receptor antagonist Compound D (dihydrochloride salt) in 18 healthy participants (mean±SEM) after administration of 250 mg Compound D as an oral solution.

FIG. 2 shows Substance P-induced venodilation expressed as percent reversal of phenylephrine-induced preconstriction after oral administration of the NK-1 receptor antagonist Compound D (dihydrochloride salt) or placebo in 17 healthy participants (mean±SEM).

FIG. 3 shows antagonism of Substance P-induced venodilation after oral administration of the NK-1 receptor antagonist Compound D (dihydrochloride salt) or placebo in 17 healthy participants (mean±SEM). Data are expressed as a percentage of the initial individual response to Substance P which was set to 100%.

FIG. 4 shows mean Substance P-induced venodilation (% antagonism) plotted against the corresponding Compound D (dihydrochloride salt) plasma concentrations.

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of pharmaceutical sciences or the art relevant to the range or element at issue. The amount of broadening from the strict numerical boundary depends upon many factors. For example, some of the factors to be considered may include the criticality of the element and/or the effect a given amount of variation will have on the performance of the claimed subject matter, as well as other considerations known to those of skill in the art. Thus, as a general matter, “about” or “approximately” broaden the numerical value. For example, in some cases, “about” or “approximately” may mean ±5%, or ±10%, or ±20%, or ±30% depending on the relevant technology. Also, the disclosure of ranges is intended as a continuous range including every value between the minimum and maximum values recited as well as any ranges that can be formable thereby.

In one embodiment, the present invention provides N-triazolylmethyl-piperazine derivative compounds. Such compounds are useful in treating or preventing, inter alia, peripheral disturbances induced by NK-1, for example functional and inflammatory disturbances of the gastrointestinal tract, including but not limited to irritable bowel syndrome (IBS) and inflammatory bowel diseases such as Crohn's disease and ulcerative colitis (collectively “NK-1 receptor mediated disorders”).

In another embodiment, compounds of the invention are those compounds of Formula I: 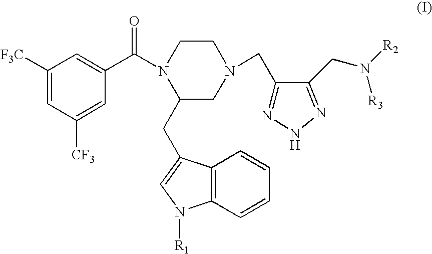
wherein

R¹ is hydrogen or lower alkyl;

R² is lower alkyl, di-lower-alkylamino lower alkyl, lower-alkoxycarbonyl lower alkyl; cyclo(hetero)alkyl having 5-6 ring atoms, which may optionally be substituted once or twice by lower alkyl and which optionally contains 1-2 double bonds; (hetero)phenyl lower alkyl optionally substituted once or twice in the (hetero)phenyl ring by halogen, lower alkyl and/or lower alkoxy, the lower-alkyl chain of which (hetero)phenyl lower alkyl is optionally substituted once or twice by lower alkyl or by spiro-C₄-C₅-alkylene; or phenyl lower alkoxy optionally substituted once or twice in the phenyl ring by halogen, lower alkyl and/or lower alkoxy; and

R³ is lower alkyl, lower-alkoxycarbonyl lower alkyl or cyclo(hetero)alkyl with 5-6 ring atoms which is optionally substituted once or twice by lower alkyl; or

R² and R³, together with the nitrogen to which they are bonded, form a cyclic group B:

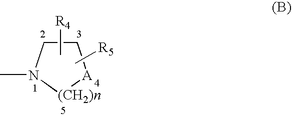

wherein A is nitrogen, oxygen, methylene or methylidene, the double bond of which, together with the adjacent carbon, is formed in position 3 of group B;

n is a whole number from 1 to 3;

R⁴ is hydrogen, lower alkyl, lower-alkoxy lower alkyl, lower alkoxycarbonyl, lower-alkoxycarbonyl lower alkyl, di-lower-alkylamino lower alkyl; (hetero)phenyl optionally substituted once or twice by halogen, lower alkyl and/or lower alkoxy; (hetero)phenyl lower alkyl optionally substituted once or twice in the (hetero)phenyl ring by halogen, lower alkyl and/or lower alkoxy, the lower-alkyl chain of which (hetero)phenyl lower alkyl is optionally substituted once or twice by lower alkyl; cyclo(hetero)alkyl with 5-6 ring atoms, or cyclo(hetero)alkyl lower alkyl, the cyclo(hetero)alkyl group of which has 5-6 ring atoms; and

R⁵ is hydrogen, lower alkyl or lower-alkoxy lower alkyl; or

R⁴ and R⁵ together are spiroethylenedioxy bonded to a carbon of group B; C₃-C₄-alkylene bonded to two adjacent atoms of group B; or phenyl fused via two adjacent carbons of group B; or

R² and R³ together with the nitrogen to which they are bonded, form a pyrrolidine ring which is substituted twice by C₄-alkylene which is bonded each time via two adjacent carbon atoms; or a physiologically compatible acid addition salts thereof.

Compounds of Formula I can be prepared by any suitable process, for example by the processes disclosed in U.S. Pat. No. 6,407,106 and U.S. Patent Publication No. 2002/0065276, each of which are hereby individually incorporated herein by reference in their entirety.

Wherein, in the compounds of Formula I, the substituents are or contain lower alkyl or alkoxy groups, such groups can be straight-chain or branched and can contain 1 to 4 carbon atoms. In one embodiment, whenever the substituents contain halogen, the halogen can be fluorine, chlorine or bromine.

Wherein, in the compounds of Formula I, the substituents are or contain cyclo(hetero)alkyl, the cyclo(hetero)alkyl may be a pure carbocyclic group, or it may also represent carbocyclic compounds in which in each case 1 to 3 ring carbon atoms are replaced by nitrogen, oxygen and/or sulfur. Nitrogen and oxygen are illlustrative heteroatoms.

Wherein, in the compounds of Formula I, the substituents are or contain (hetero)phenyl, the (hetero)phenyl can stand for phenyl, or may also represent phenyl wherein 1 to 3 ring carbon atoms are replaced by nitrogen.

In one embodiment, R¹ stands for hydrogen. In another embodiment, where R¹ stands for lower alkyl, the lower alkyl is methyl. In other embodiments, R² stands for lower alkyl, in particular methyl, ethyl, isopropyl or tertiary butyl; for di-lower-alkylamino lower-alkyl, in particular dimethylaminoethyl or dimethylamino-n-propyl; for lower-alkoxycarbonyl lower alkyl, in particular ethoxycarbonylmethyl; for cyclo(hetero)alkyl having 5 to 6 ring atoms, optionally substituted once by lower alkyl, in particular methyl, in particular for optionally substituted cyclopentyl, cyclohexyl or piperidinyl; for heterophenyl lower alkyl optionally substituted once or twice in the heterophenyl ring by lower alkyl, in particular methyl, or by lower alkoxy, in particular methoxy, in particular for optionally substituted pyridyl; or for phenyl-C₂-C₄-alkyl substituted once or twice in the phenyl ring by lower alkyl, in particular methyl, or by lower alkoxy, in particular methoxy.

In various embodiments, R³ represents lower alkyl, in particular methyl, ethyl or isopropyl; or for lower-alkoxycarbonyl lower alkyl, in particular ethoxycarbonylmethyl. In another embodiment, R² and R³ are not simultaneously isobutyl.

In various embodiments, when R² and R³, together with the nitrogen to which they are bonded, form group B, R⁴ stands for hydrogen; for lower alkyl, in particular methyl or isopropyl; for lower-alkoxy lower alkyl, in particular methoxymethyl; for lower-alkoxycarbonyl lower alkyl, in particular ethoxycarbonylmethyl; for di-lower-alkylamino lower alkyl, in particular dimethylaminoethyl; for (hetero)phenyl optionally substituted once by lower alkyl, in particular methyl, or by lower alkoxy, in particular methoxy, in particular for optionally substituted phenyl, pyridyl, pyrimidyl or pyrazolyl; for (hetero)phenyl lower alkyl optionally substituted once in the (hetero)phenyl ring by halogen, lower alkyl, in particular methyl, or by lower alkoxy, in particular methoxy, in particular for optionally substituted benzyl or pyridyl lower alkyl; for cyclo(hetero)alkyl having 5 to 6 ring atoms, in particular for cyclohexyl, pyrrolidinyl or piperidinyl; or for cyclo(hetero)alkyl lower alkyl, the cyclo(hetero)alkyl ring of which has 5 to 6 ring atoms, in particular for pyrrolidinyl-C₁-C₂-alkyl, morpholinoethyl or cyclohexylmethyl.

In various embodiments where group B is present, R⁵ stands for hydrogen; for lower alkyl, in particular methyl; or for lower-alkoxy lower alkyl, in particular methoxymethyl.

In another embodiment, where compounds of Formula I are those in which group B is present, R⁴ and R⁵ are not bonded to the same atom of group B, with the exception of the compounds of Formula I in which R⁴ and R⁵ together are spiroethylenedioxy bonded to a carbon of group B. In another embodiment, compounds of Formula I are those in which R⁴ and R⁵ are C₃-C₄-alkylene bonded to two adjacent ring atoms of group B.

In another embodiment, where group B is present, n stands for a whole number from 1 to 3. In another embodiment, where R⁴ and R⁵ are both hydrogen and at the same time A stands for methylene, n stands for 2 or 3.

Generally, the substituents R⁴ and R⁵ of group B may be bonded to each ring atom of the group, including the ring atoms formed by A, which do not stand for oxygen. Where a ring atom of group B is substituted by R⁴ and R⁵, R⁴ and R⁵ replace a hydrogen atom otherwise present at the same location, so that the usual valencies of the ring atoms of group B are retained. Where A stands for methylidene, the double bond thereof is preferably formed with the adjacent carbon in position 3 of group B, which in this case likewise forms a methylidene group.

Illustrative compounds of Formula I, in which an optionally present group B stands for pyrrolidine substituted by R⁴ and R⁵, wherein R⁴ and R⁵ are not both simultaneously hydrogen, or wherein an optionally present group B stands for 2,5-dihydropyrrole, piperidine, piperazine, morpholine or diazepan, each substituted by R⁴ and R⁵.

In one embodiment, Formula I is (2R)-1-[3,5-bis(trifluoromethyl)benzoyl]-2-(1H-indol-3-ylmethyl)-4-{[5-(morpholinomethyl)-2H-1,2,3-triazol-4-yl]methyl}piperazine or an enantiomer, isomer, tautomer thereof. Pharmaceutically acceptable salts of compounds described herein also comprise further embodiments of the invention, for example a pharmaceutically acceptable acid addition salts thereof.

“Pharmaceutically acceptable salts,” or “salts,” also include the salts of the various compounds of the invention prepared from any suitable organic or inorganic acid. In one embodiment, the acid is a sulfuric acid, phosphoric acid or hydrohalic acid, for example lower aliphatic monocarboxylic, dicarboxylic or tricarboxylic acids. In another embodiment, the acid is a sulfonic acid, for example lower alkanesulfonic acids or a benzenesulfonic acid optionally substituted in the benzene ring by halogen or lower alkyl. Illustrative pharmaceutically acceptable acids include formic, acetic, algenic, anthranilic, ascorbic, aspartic, beta.-hydroxybutyric, benzoic, benzenesulfonic, citric, cyclohexylaminosulfonic, embonic, ethanesulfonic, fumaric, galactaric, galacturonic, glutamic, glycolic, gluconic, glucuronic, 2-hydroxyethanesulfonic, hydrochloric, lactic, malic, maleic, mandelic, methanesulfonic, mesylic, propionic, pyruvic, phenylacetic, pantothenic, p-hydroxybenzoic, succinic, stearic, salicylic, sulfuric, sulfanilic, tartaric, toluenesulfonic, and tartaric acids.

In one embodiment, a composition of the invention comprises (2R)-1-[3,5-bis(trifluoromethyl)benzoyl]-2-(1H-indol-3-ylmethyl)-4-{[5-(morpholinomethyl)-2H-1,2,3-triazol-4-yl]methyl}piperazine-dihydrochloride.

The compounds of Formula I contain a chiral (asymmetric) carbon atom, namely the carbon atom bearing the 1H-indol-3-ylmethyl radical in position 2 of the piperazine parent structure. The compounds of Formula I can thus exist in several stereoisomeric forms. The present invention comprises both mixtures of optical isomers and the isomerically pure compounds of Formula I. In one embodiment, compounds of Formula I are those in which the indolylmethyl radical is located in position 2R of the piperazine ring. Formula I can be obtained from the mixtures of optical isomers by known techniques, for example by chromatographic separation on chiral separating materials or by reaction with suitable optically active acids, for example tartaric acid or 10-camphorsulfonic acid, and subsequent separation into their optically active antipodes by fractional crystallization of the resulting diastereomeric salts.

In the compounds of Formula I, the 1,2,3-triazole ring may be present in several tautomeric forms, so the hydrogen atom may be bonded to different atoms of the 1,2,3-triazole ring. Within the scope of the present invention, the compounds of Formula I jointly and individually comprise all possible tautomers of the triazole ring.

In another embodiment, the composition of the invention is Compound D or an isomer, tautomer, or pharmaceutically acceptable salt of any of the foregoing: 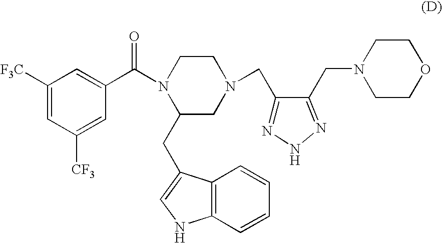
Pharmaceutical Compositions

Compounds of the invention can be prepared as pharmaceutical compositions. Such compositions of the invention can, if desired, include one or more pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition. Excipients include, by way of illustration and not limitation, diluents, disintegrants, binding agents, adhesives, wetting agents, lubricants, glidants, surface modifying agents, substances added to mask or counteract a disagreeable taste or odor, flavors, dyes, fragrances, and substances added to improve appearance of the composition.

Excipients optionally employed in compositions of the invention can be solids, semi-solids, liquids or combinations thereof. Compositions of the invention containing excipients can be prepared by any known technique of pharmacy that comprises mixing an excipient with a drug or therapeutic agent.

Compositions of the invention optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of α- and amorphous cellulose (e.g., Rexcel™) and powdered cellulose; calcium carbonate; glycine; bentonite; polyvinylpyrrolidone; and the like. Such diluents, if present, constitute in total about 5% to about 99%, about 10% to about 85%, or about 20% to about 80%, of the total weight of the composition. The diluent or diluents selected preferably exhibit suitable flow properties and, where tablets are desired, compressibility.

The use of extragranular microcrystalline cellulose (that is, microcrystalline cellulose added to a wet granulated composition after a drying step) can be used to improve hardness (for tablets) and/or disintegration time.

Compositions of the invention optionally comprise one or more pharmaceutically acceptable disintegrants as excipients, particularly for tablet formulations. Suitable disintegrants include, either individually or in combination, starches, including sodium starch glycolate (e.g., Explotab™ of PenWest) and pregelatinized corn starches (e.g., National™ 1551, National™ 1550, and Colocorn™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, xanthan, locust bean, karaya, pectin and tragacanth gums.

Disintegrants may be added at any suitable step during the preparation of the composition, particularly prior to a granulation step or during a lubrication step prior to compression. Such disintegrants, if present, constitute in total about 0.2% to about 30%, about 0.2% to about 10%, or about 0.2% to about 5%, of the total weight of the composition.

Compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™). Such binding agents and/or adhesives, if present, constitute in total about 0.5% to about 25%, about 0.75% to about 15%, or about 1% to about 10%, of the total weight of the composition.

Compositions of the invention optionally comprise one or more pharmaceutically acceptable wetting agents as excipients. Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, about 0.4% to about 10%, or about 0.5% to about 5%, of the total weight of the composition.

Compositions of the invention optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium (magnesium stearate), calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. Such lubricants, if present, constitute in total about 0.1% to about 10%, about 0.2% to about 8%, or about 0.25% to about 5%, of the total weight of the composition.

Suitable anti-adherents include talc, cornstarch, DL-leucine, sodium lauryl sulfate and metallic stearates. Talc is a anti-adherent or glidant used, for example, to reduce formulation sticking to equipment surfaces and also to reduce static in the blend. One or more anti-adherents, if present, constitute about 0.1% to about 10%, about 0.25% to about 5%, or about 0.5% to about 2%, of the total weight of the composition.

Glidants can be used to promote powder flow of a solid formulation. Suitable glidants include colloidal silicon dioxide, starch, talc, tribasic calcium phosphate, powdered cellulose and magnesium trisilicate.

Compositions of the present invention can comprise one or more anti-foaming agents. Simethicone is an illustrative anti-foaming agent. Anti-foaming agents, if present, constitute about 0.001% to about 5%, about 0.001% to about 2%, or about 0.001% to about 1%, of the total weight of the composition.

Compositions of the present invention can comprise one or more flavoring agents, sweetening agents, and/or colorants. Flavoring agents useful in the present invention include, without limitation, acacia syrup, alitame, anise, apple, aspartame, banana, Bavarian cream, berry, black currant, butter, butter pecan, butterscotch, calcium citrate, camphor, caramel, cherry, cherry cream, chocolate, cinnamon, citrus, citrus punch, citrus cream, cocoa, coffee, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, MagnaSweet®, maltol, mannitol, maple, menthol, mint, mint cream, mixed berry, nut, orange, peanut butter, pear, peppermint, peppermint cream, Prosweet® Powder, raspberry, root beer, rum, saccharin, safrole, sorbitol, spearmint, spearmint cream, strawberry, strawberry cream, stevia, sucralose, sucrose, Swiss cream, tagatose, tangerine, thaumatin, tutti fruitti, vanilla, walnut, watermelon, wild cherry, wintergreen, xylitol, and combinations thereof, for example, anise-menthol, cherry-anise, cinnamon-orange, cherry-cinnamon, chocolate-mint, honey-lemon, lemon-lime, lemon-mint, menthol-eucalyptus, orange-cream, vanilla-mint, etc. Flavoring agents, if present, constitute about 0.001% to about 25%, about 0.001% to about 15%, or about 0.001% to about 10%, of the total weight of the composition.

Sweetening agents that can be used in the present invention include, for example, acesulfame potassium (acesulfame K), alitame, aspartame, cyclamate, cylamate, dextrose, isomalt, MagnaSweet®, maltitol, mannitol, neohesperidine DC, neotame, Prosweet® Powder, saccharin, sorbitol, stevia, sucralose, sucrose, tagatose, thaumatin, xylitol, and the like. Sweetening agents, if present, constitute about 0.001% to about 50%, about 0.001% to about 20%, or about 0.001% to about 10%, of the total weight of the composition.

The foregoing excipients can have multiple roles as is known in the art. For example, starch can serve as a filler as well as a disintegrant. The classification of excipients above is not to be construed as limiting in any manner.

Pharmaceutical Dosage Forms

Compositions of the invention can be in the form of an orally deliverable dosage unit. The terms “oral administration” or “orally deliverable” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration.

Compositions of the present invention can be formulated as solid, liquid or semi-solid dosage forms suitable for oral administration. In one embodiment, such compositions are in the form of discrete dose units or dosage units. The terms “dose unit” and/or “dosage unit” herein refer to a portion of a pharmaceutical composition that contains an amount of a therapeutic agent suitable for a single administration to provide a therapeutic effect. Such dosage units may be orally administered one to a small plurality (i.e. 1 to about 4) of times per day, or as many times as needed to elicit a therapeutic response. A particular dosage form can be selected to accommodate any desired frequency of administration to achieve a specified daily dose. Typically one dose unit, or a small plurality (i.e. up to about 4) of dose units, provides a sufficient amount of the active drug to result in the desired response or effect.

Alternatively, compositions of the invention can be formulated for rectal, topical, inhalation, intranasal or parenteral (e.g. subcutaneous, intramuscular, intravenous and intradermal or infusion) delivery.

In one embodiment, compositions of the invention are suitable for rapid onset of therapeutic effect. In one embodiment, a single dosage unit, be it solid or liquid, comprises a therapeutically effective amount or a therapeutically and/or prophylactically effective amount of active ingredient, agent or drug, for example Formula I or Compound D. The term “therapeutically effective amount” or “therapeutically and/or prophylactically effective amount” as used herein refers to an amount of compound or agent that is sufficient to elicit the required or desired therapeutic and/or prophylactic response, as the particular treatment context may require.

It will be understood that a therapeutically and/or prophylactically effective amount of a drug for a subject is dependent inter alia on the body weight of the subject. A “subject” herein to which a therapeutic agent or composition thereof can be administered includes a human subject of either sex and of any age, and also includes any nonhuman animal, particularly a domestic or companion animal, illustratively a cat, dog or a horse.

In one embodiment, a dose unit of the invention comprises about 1 to about 2000 mg, about 10 to about 1500 mg, about 100 to about 1000 mg, about 150 to about 750 mg, about 200 to about 500 mg, about 225 to about 400 mg, or about 250 to about 350 mg of a compound of Formula I, for example Compound D. In another embodiment, a composition of the invention can comprise about 5 mg, about 10 mg, about 20 mg, about 30 mg, about 40 mg, about 50 mg, about 60 mg, about 70 mg, about 80 mg, about 90 mg, about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg, about 820 mg, about 840 mg, about 860 mg, about 880 mg, about 800 mg, about 820 mg, about 840 mg, about 860 mg, about 880 mg, about 900 mg, about 920 mg, about 940 mg, about 960 mg, about 980 mg, about 100 mg, about 1020 mg, about 1040 mg, about 1060 mg, about 1080 mg, about 1100 mg, about 1200 mg, about 1220 mg, about 1240 mg, about 1260 mg, about 1280 mg, about 1300 mg, about 1320 mg, about 1340 mg, about 1360 mg, about 1380 mg, about 1400 mg, about 1420 mg, about 1460 mg, about 1480 mg or about 1500 mg of a compound of Formula I.

In another embodiment, a composition of the invention is administered to a human subject in an amount sufficient to provide a daily dose of active ingredient, for example 2-indolylmethyl-piperazine derivative such as Formula D, of about 1 to about 2000 mg, about 10 to about 1500 mg, about 100 to about 1000 mg, about 150 to about 750 mg, about 200 to about 500 mg, about 225 to about 400 mg, or about 250 to about 350 mg, for example about 5 mg, about 10 mg, about 20 mg, about 30 mg, about 40 mg, about 50 mg, about 60 mg, about 70 mg, about 80 mg, about 90 mg, about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg, about 820 mg, about 840 mg, about 860 mg, about 880 mg, about 800 mg, about 820 mg, about 840 mg, about 860 mg, about 880 mg, about 900 mg, about 920 mg, about 940 mg, about 960 mg, about 980 mg, about 100 mg, about 1020 mg, about 1040 mg, about 1060 mg, about 1080 mg, about 1100 mg, about 1200 mg, about 1220 mg, about 1240 mg, about 1260 mg, about 1280 mg, about 1300 mg, about 1320 mg, about 1340 mg, about 1360 mg, about 1380 mg, about 1400 mg, about 1420 mg, about 1460 mg, about 1480 mg or about 1500 mg.

Solid Dosage Forms

In some embodiments, compositions of the invention are solid dosage forms. Non-limiting examples of suitable solid dosage forms include tablets (e.g. standard tablets, suspension tablets, bite suspension tablets, rapid dispersion tablets, chewable tablets, effervescent tablets, bilayer tablets, etc), caplets, capsules (e.g. a soft or a hard gelatin capsule), powder (e.g. a packaged powder, a dispensable powder or an effervescent powder), lozenges, buccal tablets, sublingual tablets, sachets, cachets, troches, pellets, granules, microgranules, encapsulated microgranules, powder aerosol formulations, buccal or sublingual sprays, or any other solid dosage form reasonably adapted for oral administration.

Tablets are an illustrative dosage form for compositions of the invention. Tablets can be prepared according to any of the many relevant, well known pharmacy techniques. In one embodiment, tablets or other solid dosage forms can be prepared by processes that employ one or a combination of methods including, without limitation, (1) dry mixing, (2) direct compression, (3) milling, (4) dry or non-aqueous granulation, (5) wet granulation, or (6) fusion.

The individual steps in the wet granulation process of tablet preparation typically include milling and sieving of the ingredients, dry powder mixing, wet massing, granulation and final grinding. Dry granulation involves compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). Typically, no wet binder or moisture is involved in any of the steps.

In another embodiment, solid dosage forms such as tablets can be prepared by mixing the active ingredient with at least one optional pharmaceutical excipient to form a substantially homogeneous preformulation blend. The preformulation blend can then be subdivided and optionally further processed (e.g. compressed, encapsulated, packaged, dispersed, etc.) into any desired dosage forms.

Compressed tablets can be prepared by compacting a powder or granulation composition of the invention. The term “compressed tablet” generally refers to a plain, uncoated tablet suitable for oral ingestion, prepared by a single compression or by pre-compaction tapping followed by a final compression. Tablets of the present invention may be coated or otherwise compounded to provide a dosage form affording the advantage of improved handling or storage characteristics. The term “suspension tablet” as used herein refers to a compressed tablet that rapidly disintegrates after placement in water.

Liquid Dosage Forms

In another embodiment of the invention, compositions are in the form of liquid dosage forms or units. Non-limiting examples of suitable liquid dosage forms include solutions, suspension, elixirs, syrups, liquid aerosol formulations, etc.

In another embodiment, compositions of the invention are in the form of a powder for suspension that can be suspended in a liquid vehicle prior to administration to a subject. While the powder for suspension itself can be a solid dosage form of the present invention, the powder dispersed in liquid also comprises a liquid embodiment of the invention.

In one embodiment, upon storage of a liquid composition of the invention in a closed container maintained at either room temperature, refrigerated (e.g. about 5-10° C.) temperature, or freezing temperature for a period of about 1, 2, 3, 4, 5, 6, 7, 8, 9, 10, 11, or 12 months, exhibit at least about 90%, at least about 92.5%, at least about 95%, or at least about 97.5% of the original active ingredient present therein.

Pharmacokinetics

In one embodiment, a composition of the invention is orally administered to a subject in an amount sufficient to achieve a blood plasma or serum concentration of the active compound of about 10 ng/ml to about 77 ng/ml, or at least about 20 ng/ml, at least about 30 ng/ml, at least about 40 ng/ml, at least about 50 ng/ml, at least about 60 ng/ml, at least about 70 ng/ml, or at least about 75 ng/ml at any time within about 2 hours after oral administration, within about 1.75 hours after administration, within about 1.5 hours after administration, or within about 1 hour after administration.

In another embodiment, a composition of the invention is orally administered to a subject in an amount sufficient to achieve a blood plasma or serum concentration of the active compound of about 10 ng/ml to about 77 ng/ml, about 15 ng/ml to about 70 ng/ml, or about 20 ng/ml to about 60 ng/ml at any time within about 2 hours after oral administration, within about 1.75 hours after administration, within about 1.5 hours after administration, or within about 1 hour after administration.

In another embodiment, a composition of the invention is orally administered to a subject in an amount sufficient to achieve an AUC_{0-∞} plasma or serum concentration of the compound of about 50 to about 400 h·ng/ml, about 100 to about 300 h·ng/ml, about 150 to about 250 h·ng/ml, or about 175 to about 225 h·ng/ml.

In another embodiment, a composition of the invention is orally administered to a subject in an amount sufficient to achieve at least one of: (a) a blood plasma or serum concentration of the active compound of about 10 ng/ml to about 77 ng/ml, or of at least about 20 ng/ml, at least about 30 ng/ml, at least about 40 ng/ml, at least about 50 ng/ml, at least about 60 ng/ml, at least about 70 ng/ml, or at least about 75 ng/ml at any time within about 2 hours after oral administration, within about 1.75 hours after administration, within about 1.5 hours after administration, or within about 1 hour after administration; or (b) an AUC_{0-∞} plasma or serum concentration of the compound of about 50 to about 400 h·ng/ml, about 100 to about 300 h·ng/ml, about 150 to about 250 h·ng/ml, or about 175 to about 225 h·ng/ml.

In another embodiment, a composition of the invention is orally administered to a subject in an amount sufficient to achieve: (a) a blood plasma or serum concentration of the active compound of about 10 ng/ml to about 77 ng/ml, or of at least about 20 ng/ml, at least about 30 ng/ml, at least about 40 ng/ml, at least about 50 ng/ml, at least about 60 ng/ml, at least about 70 ng/ml, or at least about 75 ng/ml at any time within about 2 hours after oral administration, within about 1.75 hours after administration, within about 1.5 hours after administration, or within about 1 hour after administration; and (b) an AUC_{0-∞} plasma or serum concentration of the compound of about 50 to about 400 h·ng/ml, about 100 to about 300 h·ng/ml, about 150 to about 250 h·ng/ml, or about 175 to about 225 h·ng/ml.

In another embodiment, a composition of the invention is orally administered to a subject in an amount sufficient to achieve a blood plasma or serum concentration of the active compound of at least one of the following: at least about 20 ng/ml at 15 minutes after dosing (i.e. the administering step), at least about 15 ng/ml at 1 hour after dosing, at least about 10 ng/ml at 2 hours after dosing, at least about 5 ng/ml at 4 hours after dosing, at least about 2 ng/ml at 8 hours after dosing and/or at least about 1 ng/ml at 12 hours after dosing.

In another embodiment, a composition of the invention is orally administered to a subject in an amount sufficient to achieve a blood plasma or serum concentration of the active compound of each of the following: at least about 20 ng/ml at 15 minutes after dosing (i.e. the administering step), at least about 15 ng/ml at 1 hour after dosing, at least about 10 ng/ml at 2 hours after dosing, at least about 5 ng/ml at 4 hours after dosing, at least about 2 ng/ml at 8 hours after dosing and at least about 1 ng/ml at 12 hours after dosing.

In another embodiment, a composition of the invention is orally administered to a plurality of subjects in an amount sufficient to achieve a ratio of maximal Cₘₐₓ to minimal Cₘₐₓ (as among the plurality of subjects) not greater than about 10, not greater than about 9, not greater than about 8, not greater than about 7, or not greater than about 6.5.

In another embodiment, a composition of the invention is orally administered to a plurality of subjects in an amount sufficient to achieve a ratio of maximal AUC_{0-∞} to minimal AUC_{0-∞} (as among the plurality of subjects) not greater than about 10, not greater than about 9, not greater than about 8, not greater than about 7, or not greater than about 6.5.

### EXAMPLES

### Example 1

The pharmacokinetics after a single oral dose of the dihydrochloride salt of Compound D ((2R)-1-[3,5-bis(trifluoromethyl)benzoyl]-2-(1H-indol-3-ylmethyl)-4-{[5-(morpholinomethyl)-2H-1,2,3-triazol-4-yl]methyl}piperazine-dihydrochloride) were assessed in healthy male volunteers. Pharmacodynamic effects were also evaluated by measuring the antagonism of Substance P-induced venodialation using the hand vein compliance technique.

Nineteen healthy male non-smokers participated in a randomized, double-blind, placebo-controlled, cross-over study after each gave written informed consent. Only healthy volunteers without concurrent drug use were included in the study. All volunteers had a physical examination, a 12-lead ECG, and a laboratory examination to exclude haematological, renal, or hepatic dysfunction. Further exclusion criteria were: a history of allergies, known conditions causing endothelial dysfunction as diabetes, hyperlipidemia, arterial hypertension, hyperhomocysteinemia, and smoking, regular medication or treatment with drugs within the last 2 weeks, acute or chronic illness, and drug or alcohol abuse.

Hand Vein Compliance Technique

The participants abstained from alcohol for at least 24 hours and from methylxanthine-containing beverages for at least 12 hours before the measurements of hand vein compliance were made. Two hours before investigations were started they had a standardized light breakfast. Venodilator responses were investigated in a quiet room maintained at a constant temperature between 23 and 25° C. using the dorsal hand vein compliance technique according to Aellig (See W. H. Aellig, A new technique for recording compliance of human hand veins. 1981. Br J Clin Pharmacol 2004; 58:S768-74) with modifications as described previously (Fricker R, et al., Endothelial venodilator response in carriers of genetic polymorphisms involved in NO synthesis and degradation. Br J Clin Pharmacol 2004; 58:169-77).

Hand vein compliance measurements started in the morning and the participants were asked to remain in a supine position throughout the study. The hand under investigation was placed on a vacuum pillow sloping upwards at an angle of 30° from horizontal. All vasoactive compounds were administered through a butterfly needle at a constant flow rate (0.25 ml·min⁻¹) into the vein under investigation. In each participant, the same hand vein was used for both study phases. Changes of the diameter of the vein were recorded using a linear variable differential transformer (Schaevitz®, Type 100 MHR, Pennsauken, N.J., USA) with a freely movable core (weight 0.5 g) resting over the centre of the vein under investigation. Transformer signals were amplified by a Schaevitz® CAS series signal conditioner and the output was recorded on a strip-chart recorder (LKB 2210 recorder, LKB Produkter AB®, Bromma, Sweden) at a paper speed of 0.5 cm·min⁻¹. The difference between the position of the core before and during inflation of a sphygmomanometer cuff on the same upper arm to 40 mmHg gave a measure of the diameter changes under a given congestion pressure. Peak heights on the strip-chart recorder were linearly proportional to the movement of the core and were measured manually in units according to standard operating procedures.

After having installed the tripod for hand vein compliance technique and having established a stable initial baseline with 4% gelatine solution defined as 100% relaxation, increasing dose-rates of the selective α₁-adrenoceptor agonist phenylephrine (Neo-Synephrine®, Abbott Laboratories, North Chicago, USA; dosages: 1.25−≦8000 ng·min⁻¹) were locally infused to constrict the vein by about 80%. This preconstriction baseline was defined as 0%, and the effect of subsequently administered vasodilators was expressed in percentage changes from the difference between the initial baseline diameter during normal saline and the diameter during stable preconstriction.

Once preconstriction was stable, Substance P (Calbiochem/Novabiochem AG, Läufelfingen, Switzerland) was co-administered until the maximal venodilation was reached (approximately 7-10 minutes). To prevent the peptide from sticking to tubing and syringes, substance P was dissolved in a 4% gelatine solution. Based on the experience of previous experiments in this setting, a Substance P dose rate of (1.5 pmol/min) was selected. If a participant reacted with less than 50% venodilation to the dose of 1.5 pmol/min, the dose was doubled to 3 pmol/min.

Next, Compound D as described herein (250 mg and 25 mg quinine sulphate) or placebo (25 mg quinine sulphate), both dissolved in water for injection and mint syrup, were administered as an oral solution. Quinine was added for blinding to mimic the bitter taste of Compound D and mint syrup to disguise the slightly yellowish colour as well as bitter taste. The infusion of Substance P (same dose as before study drug administration) was repeated at the following time points: 0.5 h, 1.25 h, 2 h, 2.75 h, 3.5 h, and 4.25 h after dosing. Each peptide application was separated by a wash-out phase of 45 min to avoid the occurrence of tolerance.

Before the end of the experiment, immediately following the last Substance P infusion, a single high dose (2 μg/min) of the vasodilator sodium nitroprusside (SNP) (Nipruss®, Schwarz Pharma AG, Monheim, Germany) was administered into the hand vein for at least 6 minutes, to demonstrate that the vein was still fully responsive and that full vasodilation could still be achieved.

Dose-rates administered locally into the hand vein were intended not to result in any systemic effects which were monitored by repeated measurements of heart rate and blood pressure. Blood pressure was taken before and after every infusion of drugs or solvents (sodium chloride, phenylephrine, Substance P); a 12-lead ECG was monitored continuously up to the end of the hand vein compliance measurements.

Venous blood samples for Compound D were taken 0.25 h before as well as 0.25 h, 0.5 h, 0.75 h, 1 h, 1.25 h, 2.0 h, 2.75 h, 3.5 h, 4.25 h, 6 h, 8 h, 12 h, and 24 h after administration. Blood was drawn into vials containing dry heparin, immediately stored on ice (+4° C.) and plasma was separated within 30 min at 3500 rpm for 10 min. The samples were stored at −20° C. until analysis. When time points of pharmacodynamic (hand vein compliance method) and pharmacokinetic measurements coincided the pharmacodynamic measurements were first finished before blood samples were taken, accepting a delay for pharmacokinetic sampling of about 5 min.

The plasma samples were then analyzed by extracting Compound D and its internal standard from plasma with diethylether, concentration and injection into an HPLC system with MS/MS detection. Accuracy and precision were within specifications; bias was <12%; the inter-day coefficient of variation was <14%. The lower limit of quantification was set at 0.2 ng/ml.

Safety was assessed by measuring ECG, pulse rate, blood pressure, haematology, blood chemistry, urinalysis, and by occurrence of adverse events.

Source verification of all data documented in case report forms was performed by an independent clinical monitor. Nineteen individuals were randomized and thus included in the safety analysis. Only the randomized participants who completed both dosing sessions according to protocol were included in the pharmacodynamic analysis (n=17). Two participants had to be withdrawn due to methodological problems during hand vein measurements. One of these two was exposed to Compound D and complete pharmacokinetic data were obtained leading to data sets of 18 participants for pharmacokinetic analysis.

The effect of Compound D was expressed as % antagonism of Substance-P induced venodilation, calculated as follows:

$$\% antagonism = 100 \% - \frac{SP - PC}{SP 0 - PC} \cdot 100$$

with SP=Substance P-induced venodilation [units], SP₀=initial Substance P-induced venodilation (pre-dosing) [units], and PC=preconstriction baseline [units].

The area under the effect-time curve (AUCₑ) was calculated according to the trapezoidal rule. Statistical analysis of AUCₑ was performed using a mixed model analysis of variance (ANOVA) including the factors subject, sequence, period, and treatment. Pharmacokinetic calculations were performed using WinNonlin Professional 4.0.1 for Windows (Pharsight Corporation, Mountain View, Calif. 94040, USA). Differences in vital signs and dose-rates were assessed with Wilcoxon signed rank test, unless stated otherwise. Data are expressed as mean±SEM. A p-value of less than 0.05 was considered significant.

Results

The participants had a mean age of 25±1 years (range 19 to 32 years), a mean weight of 78.0±1.8 kg (range 68.5 to 95.8 kg), a mean height of 183±2 cm (range 171 to 197 cm), and a mean body mass index of 23.4±1.4 kg/m² (range 21.1 to 26.0 kg/m²).

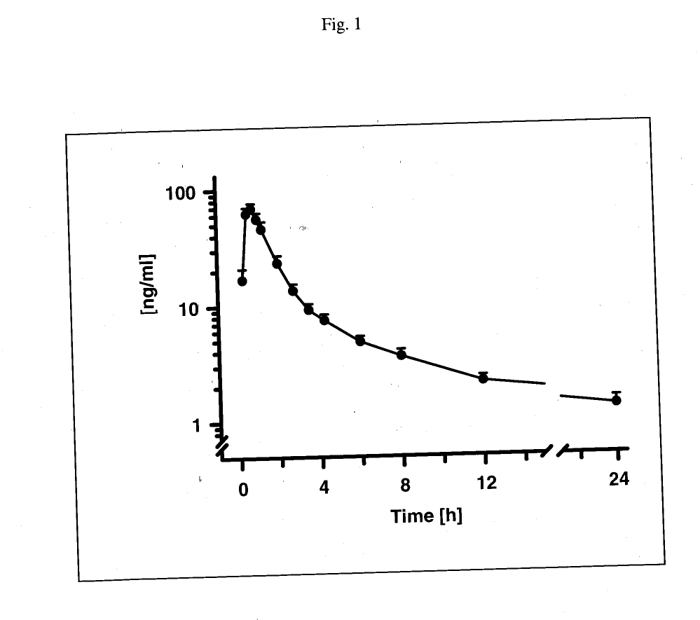

After oral administration, Compound D was rapidly absorbed and plasma concentrations reached peaks of 77±9 ng/ml within 47±3 min (FIG. 1). The mean AUC_{0-∞} was 183±22 h·ng/ml; the mean half-life was 9.9±1.6 h. In the terminal phase of concentration-time curves 24 h after dosing, Compound D was still detectable in low concentrations in all participants. Inter-individual variability was observed for Cₘₐₓ and AUC, with the ratio between maximal and minimal value being 6.5 for Cₘₐₓ and 6.3 for AUC.

Phenylephrine dose-rates used to preconstrict hand veins were similar in both study phases (Compound D 1370±297 ng/min; placebo 1491±286 ng/min; p=0.75) as was the preconstriction expressed as a percentage from the initial vein diameter recorded during infusion of solvent (Compound D 21±2%; placebo 25±4%; p=0.81). Substance P dose-rates were equal for both study treatments (Compound D 2.0±0.2 ng/min; placebo 2.1±0.2 ng/min; p=1.00 for the sign test) and the mean venodilation induced by Substance P was similar immediately before oral administration of Compound D (56±8%) or placebo (49±6%; p=0.64).

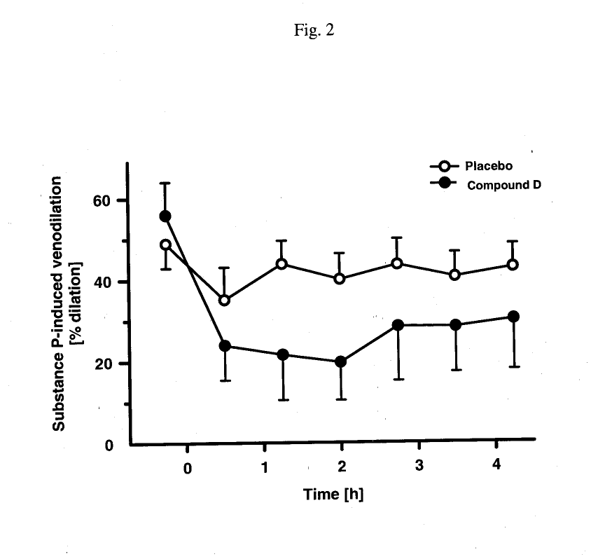

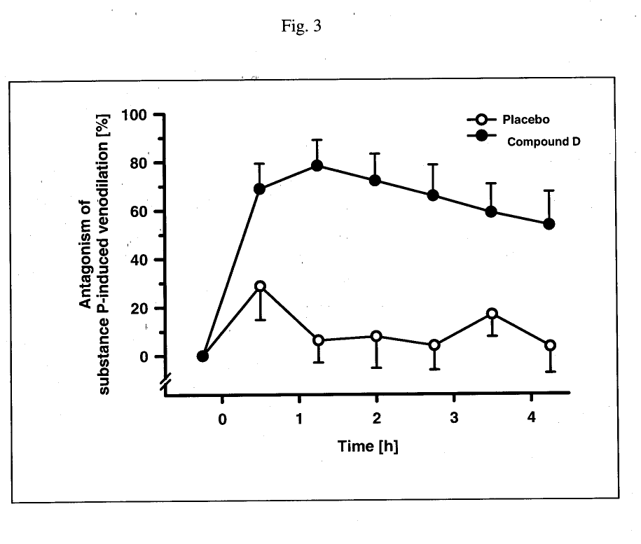

After administration of 250 mg Compound D, substance P-induced venodilation markedly decreased while vasodilation during placebo was unchanged (p<0.001; FIG. 2). The maximum antagonizing effect of Compound D averaged 95±8% (95% CI=[78; 111] and was observed after 1.47±0.24 h (median 1.25 h; 95% CI=0.96; 1.98]; FIG. 3). Correspondingly, the mean AUCₑ after administration of Compound D (278±67%·h; 95% CI=[198; 358]) was significantly higher compared to placebo (49±12%·h; 95% CI=[−24; 122]; p<0.001). There were no carry-over effects (p=0.33) and no period effects (p=0.22) as tested with ANOVA. The response to SNP at the end of the experiment was pronounced and not different for both study treatments (Compound D 92±9%; placebo 96±7%; p=0.69).

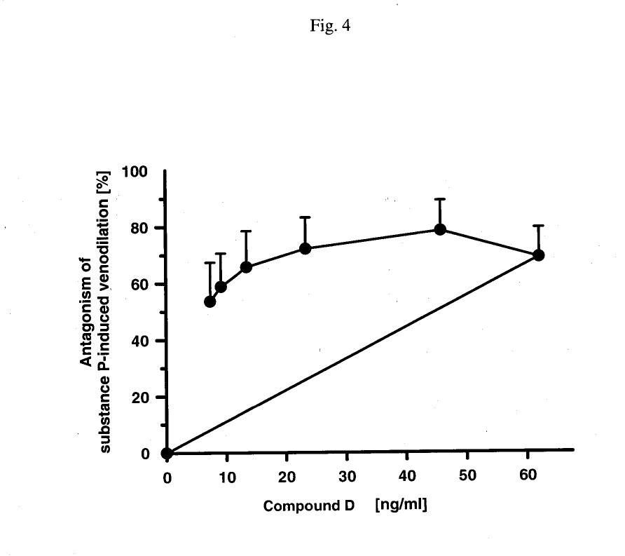

FIG. 4 shows mean Substance P-induced venodilation (% antagonism) plotted against the corresponding Compound D plasma concentrations. High Compound D plasma concentrations and maximum effects were reached already at the time of the first pharmacodynamic assessment and the antagonistic effect persisted throughout the study while plasma concentrations declined (counter-clockwise hysteresis).

Oral administration of 250 mg Compound D was well tolerated in all participants. Neither Compound D nor placebo induced significant changes in heart rate, blood pressure, or ECG parameters. At baseline and 4.5 h after administration of Compound D, blood pressure values (systolic/diastolic) were 124±3/68±3 versus 127±2/72±2 mmHg (p=0.10/p=0.12) and heart rate was 60±2 versus 63±2 beats/min (p=0.14). The respective values after placebo administration were 125±3/69±2 versus 127±2/71±2 mmHg (p=0.14/p=0.17) and 62±3 versus 63±2 beats/min (p=0.64).

No serious adverse event occurred. Six adverse events during placebo (mild headache (n=4), cloudy urine, mild orthostatic dysregulation) and 3 adverse events during Compound D (mild headache, severe headache, cloudy urine) were reported and classified as possibly related to the study drug. All resolved without any sequelae within the following hours. Cardiovascular, laboratory, and physical investigations showed no clinically relevant changes.

This study demonstrates that the neurokinin-1 receptor antagonist Compound D is an orally active and highly effective antagonist of Substance P-induced effects in humans.

What is claimed is:

1. A method for treating or preventing an NK-1 receptor mediated disorder in a human subject in need thereof, comprising the steps of:
(a) providing a pharmaceutical composition comprising a compound of Formula (I) or a pharmaceutically acceptable salt thereof 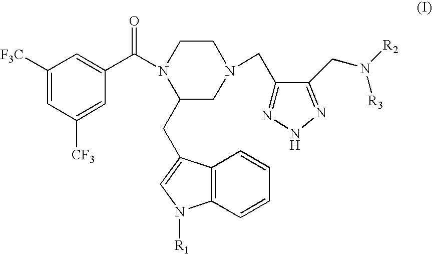
;and
(b) orally administering the composition to the subject in an amount sufficient to achieve a blood serum concentration of the compound of at least about 10 ng/ml within at least about 2 hours after oral administration; wherein:
(i) R¹ is hydrogen or lower alkyl,
(ii) R² is lower alkyl, di-lower-alkylamino lower alkyl, lower-alkoxycarbonyl lower alkyl; cyclo(hetero)alkyl having 5-6 ring atoms, which may optionally be substituted once or twice by lower alkyl and which optionally contains 1-2 double bonds; (hetero)phenyl lower alkyl optionally substituted once or twice in the (hetero)phenyl ring by halogen, lower alkyl and/or lower alkoxy, the lower-alkyl chain of which (hetero)phenyl lower alkyl is optionally substituted once or twice by lower alkyl or by spiro-C₄-C₅-alkylene; or phenyl lower alkoxy optionally substituted once or twice in the phenyl ring by halogen, lower alkyl and/or lower alkoxy, and
(iii) R³ is lower alkyl, lower-alkoxycarbonyl lower alkyl or cyclo(hetero)alkyl with 5-6 ring atoms which is optionally substituted once or twice by lower alkyl.

2. The method of claim 1 wherein R² and R³ together with the nitrogen to which they are bonded, form a cyclic group B: 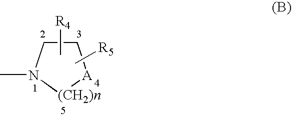
wherein
(a) A is nitrogen, oxygen, methylene or methylidene, the double bond of which, together with the adjacent carbon, is formed in position 3 of group B;
(b) n is a whole number from 1 to 3;
(c) R⁴ is hydrogen, lower alkyl, lower-alkoxy lower alkyl, lower alkoxycarbonyl, lower-alkoxycarbonyl lower alkyl, di-lower-alkylamino lower alkyl; (hetero)phenyl optionally substituted once or twice by halogen, lower alkyl and/or lower alkoxy; (hetero)phenyl lower alkyl optionally substituted once or twice in the (hetero)phenyl ring by halogen, lower alkyl and/or lower alkoxy, the lower-alkyl chain of which (hetero)phenyl lower alkyl is optionally substituted once or twice by lower alkyl; cyclo(hetero)alkyl with 5-6 ring atoms, or cyclo(hetero)alkyl lower alkyl, the cyclo(hetero)alkyl group of which has 5-6 ring atoms; and
(d) R⁵ is hydrogen, lower alkyl or lower-alkoxy lower alkyl.

3. The method of claim 2 wherein R⁴ and R⁵ together are spiroethylenedioxy bonded to a carbon of group B; C₃-C₄-alkylene bonded to two adjacent atoms of group B; or phenyl fused via two adjacent carbons of group B.

4. The method of claim 2 wherein R² and R³ together with the nitrogen to which they are bonded, form a pyrrolidine ring which is substituted twice by C₄-alkylene which is bonded each time via two adjacent carbon atoms.

5. The method of claim 1 wherein the compound comprises (2R)-1-[3,5-bis(trifluoromethyl)benzoyl]-2-(1H-indol-3-ylmethyl)-4-{[5-(morpholinomethyl)-2H-1,2,3-triazol-4-yl]methyl}piperazine-dihydrochloride.

6. The method of any one of claims 1-5 wherein step (b) comprises orally administering the composition to the subject in an amount sufficient to: (i) achieve a blood serum concentration of the compound of at least about 50 ng/ml within at least about 2 hours after oral administration; or (ii) achieve an AUC_{0-∝} plasma concentration of the compound of about 100 to about 300 h·ng/ml.

7. The method of any one of claims 1-5 wherein step (b) comprises orally administering the composition to the subject in an amount sufficient to achieve a blood serum concentration of the compound of at least about 50 ng/ml within at least about 2 hours after oral administration.

8. The method of any one of claims 1-5 wherein step (b) comprises orally administering the composition to the subject in an amount sufficient to achieve a blood serum concentration of the compound of at least about 75 ng/ml within at least about 2 hours after oral administration.

9. The method of any one of claims 1-5 wherein step (b) comprises orally administering the composition to the subject in an amount sufficient to achieve an AUC_{0-∝} plasma concentration of the compound of about 100 to about 300 h·ng/ml.

10. The method of any one of claims 1-5 wherein step (b) comprises orally administering the composition to the subject in an amount sufficient to achieve an AUC_{0-∝} plasma concentration of the compound of about 150 to about 250 h·ng/ml.

11. The method of any one of claims 1-5 wherein step (b) comprises orally administering the composition to the subject in an amount sufficient to achieve a blood plasma concentration of the active compound of at least one of the following: at least about 20 ng/ml at 15 minutes after the administering step, at least about 15 ng/ml at 1 hour after the administering step, at least about 10 ng/ml at 2 hours after the administering step, at least about 5 ng/ml at 4 hours after the administering step, at least about 2 ng/ml at 8 hours after the administering step, and/or at least about 1 ng/ml at 12 hours after the administering step.

12. The method of any one of claims 1-5 wherein step (b) comprises orally administering the composition to the subject in an amount sufficient to achieve a blood plasma concentration of the active compound of each of the following: at least about 20 ng/ml at 15 minutes after the administering step, at least about 15 ng/ml at 1 hour after the administering step, at least about 10 ng/ml at 2 hours after the administering step, at least about 5 ng/ml at 4 hours after the administering step, at least about 2 ng/ml at 8 hours after the administering step, and/or at least about 1 ng/ml at 12 hours after the administering step.

13. The method of any of claims 1-12 wherein the compound of Formula (I) is present in the composition in an amount of about 150 to about 500 mg.

14. The method of any of claims 1-12 wherein the compound of Formula (I) is present in the composition in an amount of about 250 to about 400 mg.

15. A pharmaceutical composition comprising a therapeutically effective amount of (2R)-1-[3,5-bis(trifluoromethyl)benzoyl]-2-(1H-indol-3-ylmethyl)-4-{[5-(morpholinomethyl)-2H-1,2,3-triazol-4-yl]methyl}piperazine-dihydrochloride and at least one pharmaceutically acceptable excipient.

16. The composition of claim 15 wherein the (2R)-1-[3,5-bis(trifluoromethyl)benzoyl]-2-(1H-indol-3-ylmethyl)-4-{[5-(morpholinomethyl)-2H-1,2,3-triazol-4-yl]methyl}piperazine-dihydrochloride is present in an amount of about 100 to about 500 mg.

17. The composition of claim 16 wherein the composition comprises an orally deliverable dosage form.

18. The composition of claim 17 wherein the dosage form is a solid dosage form.

19. The composition of claim 18 wherein the solid dosage form is a tablet and the at least one pharmaceutically acceptable excipient comprises a lubricant.

20. The composition of claim 17 wherein the dosage form is a liquid and the composition further comprises water.

21. The composition of claim 20 further comprising a flavoring agent, a sweetener, or a taste masking agent.

22. The composition of claim 21 wherein the flavoring agent comprises mint syrup.
